(19) United States
(12) Patent Application Publication
(10) Pub. No.: US 2025/0121127 A1
(43) Pub. Date: Apr. 17, 2025

(54) ENDOLUMINAL TREATMENT DEVICES AND RELATED METHODS

(71) Applicant: Boston Scientific Scimed Inc., Maple Grove, MN (US)

(72) Inventors: Rahul PRABHU, Brighton, MA (US); Jonathan ROOT, Townsend, MA (US); Martyn G. FOLAN, Co. Galway (IE); Emma HORGAN, Galway (IE)

(73) Assignee: Boston Scientific Scimed Inc., Maple Grove, MN (US)

(21) Appl. No.: 18/911,998

(22) Filed: Oct. 10, 2024

Related U.S. Application Data
(60) Provisional application No. 63/589,481, filed on Oct. 11, 2023.

(51) Int. Cl. A61M 1/00

(52) U.S. Cl. CPC A61M 1/90; A61M 2205/3331

(57) ABSTRACT

A distal end of a medical device is disclosed herein. The distal end including a tube. The distal end including a sponge positioned at a distal end of the tube. The sponge including a first flange, a second flange, and a saddle portion positioned between the first flange and the second flange.

### CROSS-REFERENCE TO RELATED APPLICATIONS

This application claims the benefit of priority of U.S. Provisional Patent Application No. 63/589,481 filed Oct. 11, 2023, the entirety of which is incorporated herein by reference.

### TECHNICAL FIELD

Various aspects of this disclosure relate generally to minimally invasive medical devices and methods. In particular, aspects of the disclosure relate to medical devices and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Patients with perforations, post-surgical leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices of the current disclosure may rectify some of the deficiencies described above or address other aspects of the art.

### SUMMARY

Each of the aspects disclosed herein may relate to devices and methods for endoluminal wound treatment and may include one or more of the features described in connection with any of the other disclosed aspects.

The disclosure includes medical devices and related methods useful for EVAC procedures. For example, the disclosure includes a distal end of a medical device comprising a tube and a sponge positioned at a distal end of the tube. The sponge may include a first flange, a second flange, and a saddle portion positioned between the first flange and the second flange. The saddle portion may be a first saddle portion. The sponge may include a third flange between the first flange and the second flange. The first saddle portion may be between the first flange and the third flange. The sponge may further include a second saddle portion between the third flange and the second flange. A diameter of the first saddle portion may be smaller than diameters of each of the first flange, the second flange, and the third flange. A diameter of the second saddle may be smaller than diameters of each of the first flange, the second flange, and the third flange. An outer surface of the first saddle portion taper radially inward in a distal direction. A diameter of the first flange may be larger than a diameter of the second flange. The diameter of the first flange may be larger than a diameter of the third flange. The diameter of the second may be approximately equal to the diameter of the third flange. The sponge may include a rounded portion extending distally from the second flange. One of the first flange or the second flange may be more flexible than the other of the first flange or the second flange. At least one of the first flange or the second flange may have an outer surface that tapers radially inward in a distal direction. The distal end may include a distal tip having an outer surface that tapers radially inward in the distal direction. A distalmost end of the distal tip may be pointed. A same line may extend along outer surfaces of the first flange, the second flange, and the distal tip. The first flange and the second flange may be connected to form a helix. A single curve may extend along a first surface surfaces and a second surface of the helix.

The disclosure includes a distal end of a medical device comprising a tube and a sponge positioned at a distal end of the tube. The sponge may include a flange and a portion extending distally of the flange. An outer surface of the portion extending distally of the flange may taper radially inward in a distal direction. The flange may be a first flange. The distal end may further comprise a second flange that may be distal of the first flange. The outer surface of the portion extending distally of the first flange tapers between the first flange and the second flange. The outer surface of the portion extending distally of the first flange may have a rounded shaped, forming a rounded distalmost end of the sponge. The outer surface of the portion extending distally of the first flange may be an outer surface of the second flange. A saddle portion may extend between the first flange and the second flange.

The disclosure also includes a medical method. The medical method may comprise moving a sponge coupled to a distal end of a tube proximally, such that the sponge transitions from a first configuration, in which a first flange and a second flange of the sponge may be within a cavity of a body lumen and a third flange of the sponge may be outside of the cavity, to a second configuration, in which the first flange may be within the cavity of the body lumen, and the second flange and the third flange may be outside of the cavity. The medical method may further comprise applying a negative pressure to the sponge using the tube.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts a distal end of an exemplary medical device.

FIG. 2 depicts the distal end of the medical device of FIG. 1 positioned in a target site.

FIG. 3A depicts a distal end of an exemplary medical device.

FIG. 3B depicts a cross section of the medical device of FIG. 3A.

FIG. 4A depicts a distal end of an exemplary medical device.

FIG. 4B depicts a cross section of the medical device of FIG. 4A.

FIG. 5 depicts a cross section of a distal end of an exemplary medical device.

FIG. 6A depicts a distal end of an exemplary medical device in a first configuration.

FIG. 6B depicts the distal end of the medical device of FIG. 6A in a second configuration.

FIG. 7 depicts a cross section of a distal end of an exemplary medical device.

FIG. 8 depicts a distal end of an exemplary medical device.

### DETAILED DESCRIPTION OF THE FIGURES

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. The term “diameter” also includes widths of elements that are not circular or do not have a circular cross-section.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of +10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a nasogastric tube having a sponge/foam (e.g., vacuum sealed foam) at its distal end. A proximal end of the tube may be connected to a collection container. The foam is placed endoscopically into the perforation, leak, or other wound. In some examples, EVAC includes endoluminal placement of a foam or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the material can also be in other organs reachable via the GI tract (e.g., colon). Negative pressure then is applied.

The foam in the wound, along with the negative pressure, may accelerate healing by encouraging local tissue granulation at a wound site. The foam may be replaced with increasing smaller sizes of foam as the wound heals and closes. Devices and systems suited for EVAC are limited. For example, EVAC typically requires a foam to be replaced every 3 to 5 days to reduce the risk of tissue ingrowth. Furthermore, infection of the wound may occur, which may prolong treatment of the wound. It should be understood that the terms “sponge” and “foam” as used in this disclosure are interchangeable with one another and refer to the same structure.

Presently, physicians manually shape (e.g., cut) a sponge, such as a Granufoam™ sponge and manually suture it to a vacuum tube (e.g., a nasogastric tube). Such a process may introduce multiple sources of uncertainty for a physician, particularly if the physician is less experienced in performing EVAC. The physician may be unsure of how large or small to make the sponge to accommodate a target cavity/site, and the physician may be unsure of how deep within the target cavity/site to place the sponge.

Aspects of this disclosure include devices and methods for sponges having various shapes. The sponges may be attached to a vacuum tube. Some of the disclosed sponges may include one or more flanges separated by one or more middle portions of a lesser diameter than a diameter of the one or more flanges. The one or more flanges and one or more middle portions may be retractable to create space for the wound to heal. Some sponges may narrow from the proximal end of the sponge to the distal end of the sponge. Other sponges may include specific shapes such as rounded distal ends or pointed distal ends to achieve desired patient outcomes. Any features of the sponges may be combined in any suitable manner.

Reference will now be made in detail to examples of the present disclosure described above and illustrated in the accompanying drawings. Wherever possible, the same reference numbers will be used throughout the drawings to refer to the same or like parts.

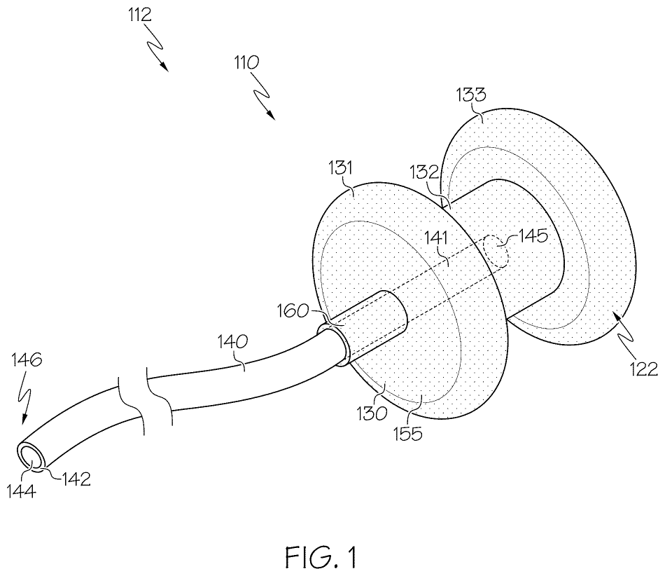

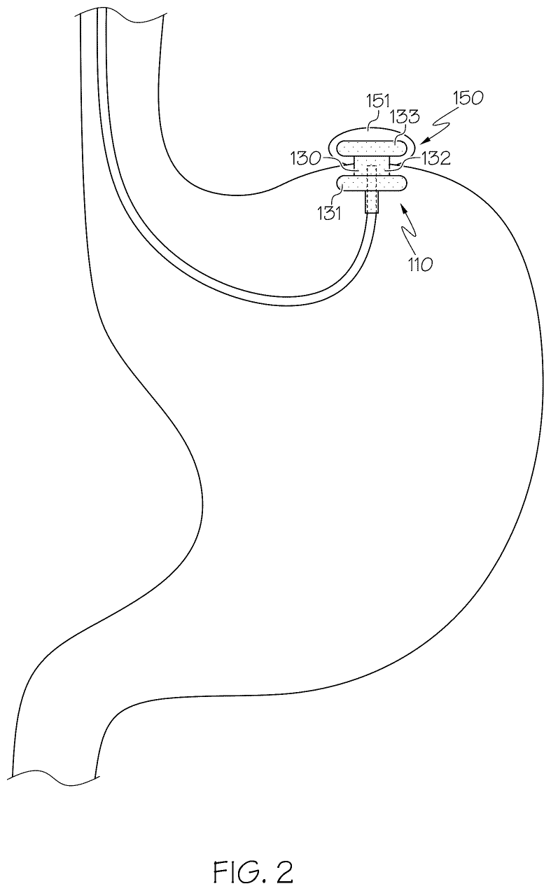

FIGS. 1 and 2 illustrate a distal end 112 of a medical device 110 in accordance with an example of this disclosure. In examples, device 110 may be utilized to perform an EVAC procedure. Device 110 may be inserted into a patient for treatment of wounds using negative pressure via a vacuum. Device 110 generally includes a foam or sponge 130 (or other mesh-like material or porous body) and a vacuum tube 140. Sponge 130 may be formed from Granufoam™ or any other suitable material.

Sponge 130 may be attached to a distal end 141 of vacuum tube 140. Vacuum tube 140 may include an outer wall 142 defining one or more lumens 144. Lumen 144 may be open at both a proximal end 146 and distal end 141 of vacuum tube 140. Outer wall 142 may optionally include additional openings (e.g., around a circumference of the distal end 141 of vacuum tube 140 and in fluid communication with lumen 144), which may increase the flow or fluid or material into lumen 144, as disclosed herein. Sponge 130 may cover all of the holes/openings of distal end 141 of vacuum tube 140. Sponge 130 may surround an entire circumference of vacuum tube 140.

Distal end 141 of vacuum tube 140 may be attached to sponge 130 via sutures, heat bond, an adhesive, a connector (e.g., a quick-connect connector) or the like. In one example, a recess may be provided in sponge 130 to receive the distal end 141 of vacuum tube 140. Vacuum tube 140 may be attached within the recess of sponge 130, which may provide additional structural support between sponge 130 and vacuum tube 140. Alternatively, sponge 130 may be extruded or cast on vacuum tube 140 as described herein.

Sponge 130 may include openings 122 throughout sponge 130. Openings 122 may be any hole, pore, or channel which provide continuous access to interconnecting channels and pores throughout sponge 130. Openings 122 may include different sizes and shapes, and may be selected based on a location of treatment within the body. For example, openings 122 may be spherical, cuboidal, irregular, or any other shape. The size of openings 122 may be approximately 50 μm to approximately 1 mm in diameter.

A proximal end 146 of vacuum tube 140 may be connected to a vacuum source (not shown), which may supply a negative pressure to sponge 130 and/or the distal end 141 of vacuum tube 140. For example, a negative pressure of approximately 125 mm Hg, or approximately 2.5 pounds per square inch (PSI), may be supplied to sponge 130. Other suitable amounts of negative pressure may be used. This negative pressure may pull fluid, material, and/or other debris into lumen 144 of vacuum tube 140 via openings 122, distal opening 145 of vacuum tube 140, and/or other openings of distal end 141, which may promote healing of a wound/cavity 151 at a target site 150.

Sponge 130 may include a first flange 131 (which may be a proximal flange), a second flange 133 (which may be a distal flange), and a middle portion 132 (e.g., a saddle). Sponge 130 may have a dumbbell shape. Middle portion 132 may be positioned between first flange 131 and second flange 133. Middle portion 132, first flange 131, and second flange 133 may be arranged along a longitudinal axis (e.g., along a central longitudinal axis) of vacuum tube 140. In some examples, first flange 131, middle portion 132, and second flange 133 may have central longitudinal axes that are approximately coaxial with one another and/or approximately coaxial with a central longitudinal axis of vacuum tube 140.

Middle portion 132 may have a cylindrical shape; however, middle portion 132 is not limited to a particular shape. In alternatives, Middle portion 132 may be any prismatic shape. In other alternatives, middle portion 132 may have a convex shape (bulge radially outward) or have a concave shape (similar to a hyperboloid). In further alternatives, Middle portion 132 may have a tapered shape, having a larger width/diameter near first flange 131 and a smaller width/diameter near second flange 133. A diameter/width of middle portion 132 may be smaller than a diameter/width of first flange 131 and/or second flange 133. In some aspects, first flange 131 and second flange 133 may have the same diameter/width. In other aspects, first flange 131 may have a different (larger or smaller) diameter than second flange 133.

First flange 131 may include a proximal facing surface 155. Sponge 130 may include a proximally extending protrusion 160 that protrudes from proximal facing surface 155. Protrusion 160 may be a tube configured to cover/surround a portion of tube 140. Protrusion 160 may have a smaller diameter/width than flanges 131, 133 and middle portion 132.

In some aspects, sponge 130 may have a solid cross-section, except for the recess for receiving distal end 141 of tube 140. For example, except for the recess for receiving distal end 141, sponge 130 may have a solid cross-section in each of first flange 131, middle portion 132, and second flange 133. In other examples, sponge 130 may have portions with a hollow center, to accommodate insertion into narrow cavities 151. In such examples, sponge 130 may include a wall that includes double walled portions at first flange 131 and second flange 133 (i.e., the wall may fold back on itself at first flange 131 and second flange 133). In such examples, first flange 131 and second flange 133 may include a hollow torus shape or a portion of the hollow torus shape. In such examples, walls of sponge 130 may be connected to one another at a distal end of sponge 130 to form a closed distal end. For example, a distal wall of second flange 133 may extend across an entire diameter of sponge 130 to form a closed distal end. In other words, one or more of first flange 131, middle portion 132, and second flange 133 may be hollow. One or more of first flange 131 and second flange 133 may be a hollow, torus-like shape. More proximal portions of sponge 130 may be hollow. Protrusion 160 may be sealed around distal end 141 of tube 140. In some examples, pores 122 of second flange 133, pores 122 of middle portion 132, and pores 122 of first flange 131 may be of different sizes/diameters. For example, pores 122 of middle portion 132 may be larger than pores 122 of first flange 131 and pores 122 of second flange 133. Pores 122 may be preferentially exposed. On initial placement of sponge 130 within cavity 151 at target site 150, pores 122 of first flange 131, middle portion 132, and second flange 133 may be exposed and subject to drainage of cavity 151. After a predetermined amount of time, cavity 151 may reduce in size or properties of the cavity 151 may otherwise change. As cavity 151 reduces in size, second flange 133 may fold over or be folded over (e.g. by a medical profession) middle portion 132 to accommodate a smaller cavity 151 and to halt/cease contact between pores 122 of middle portion 132 with cavity 151 (e.g. the surrounding tissue). Second flange 133 may be sized and shaped to fully or partially cover middle portion 132 when second flange 133 is folded over middle portion 132. The relatively smaller pores 122 of second flange 133 (e.g. the pores at a distal face of second flange 133) may replace the relatively larger pores 122 of middle portion 132. Replacing the larger pores 122 of middle portion 132 with the smaller pores 122 of second flange 133 may allow continued drainage but with a lower risk of tissue ingrowth over time. Tissue ingrowth may be a greater risk for the larger pores 122 of middle portion 132.

Flanges 131, 133 may serve as indicator for how deep to insert sponge 130 into target cavity 151. As shown in FIG. 2, flange 131 may remain outside of (e.g., proximal of) target cavity 151, and flange 133 may be disposed within target cavity 151. Such a positioning of flanges 131, 133 may help to prevent migration of sponge 130 out of cavity 151. For example, flange 133 may be an anti-migration structure/feature.

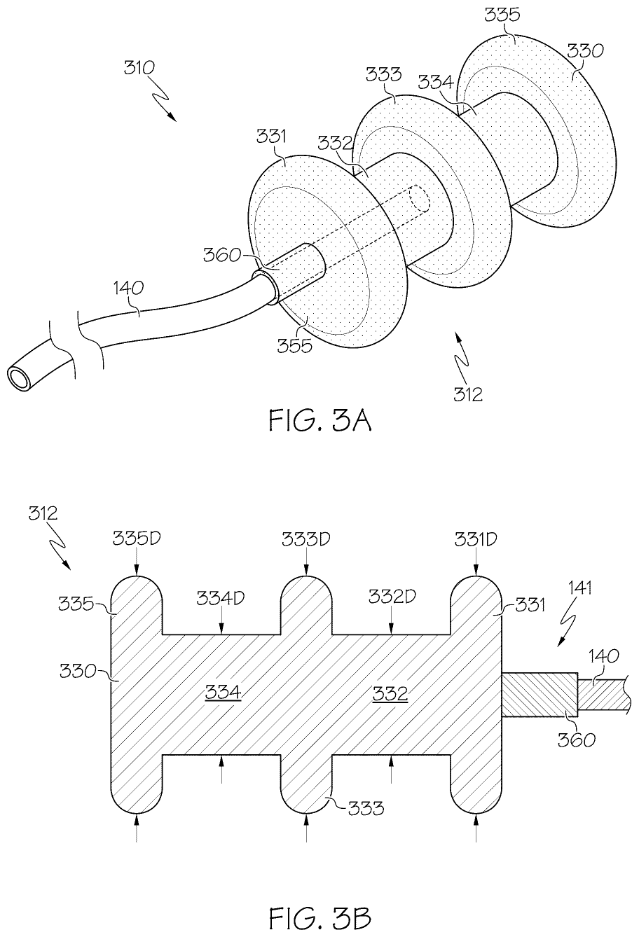

FIGS. 3A-3B depict a distal end 312 of an exemplary medical device 310. Distal end 312 may have any of the features of distal end 112, unless otherwise specified. Where feasible, reference numbers of distal end 312 add 200 to reference numbers of distal end 112 to indicate corresponding structures.

Distal end 312 may include a sponge 330 and tube 140. Sponge 330 may be positioned at distal end 141 of tube 140. Sponge 330 may include a first (proximal) flange 331, a second (middle) flange 333, and a first middle portion 332 (e.g., a first saddle). First middle portion 332 may be positioned between first flange 331 and second flange 333. Sponge 330 may further include a third (distal) flange 335 and a second middle portion 334 (e.g., a second saddle). Second flange 333 may be between first flange 331 and third flange 335. Second middle portion 334 may be positioned between second flange 333 and third flange 335. First flange 331 may be positioned proximally of second flange 333 and third flange 335, such that first flange 331 is a proximalmost flange of sponge 330. Third flange 335 may be positioned distally of first flange 331 and second flange 333, such that third flange 335 is a distalmost flange. Second flange 333 may be positioned between first flange 331 and third flange 335. First flange 331, second flange 333, third flange 335, first middle portion 332, and second middle portion 334 may be arranged along a longitudinal axis (e.g., a central longitudinal axis) of vacuum tube 140. In some examples, central longitudinal axes of flanges 331, 333, 335 and middle portions 332, 334 may be coaxial with a central longitudinal axis of vacuum tube 140.

Referring to FIG. 3B, a diameter 332D of first middle portion 332 may be smaller than a diameter 331D of first flange 331, a diameter 333D of second flange 333, and/or a diameter 335D of third flange. A diameter 334D of second middle portion 334 may be smaller than diameter 331D of first flange 331, diameter 333D of second flange 333, and/or diameter 335D of third flange. Diameter 332D and diameter 334D may be the same or approximately the same. Diameters 331D, 333D, and/or 335D may be the same or approximately the same.

Similarly to middle portion 132, first middle portion 332 and second middle portion 334 may be approximately cylindrical in shape; however first middle portion 332 and second middle portion 334 are not limited to a particular shape. In some aspects, first middle portion 332 and/or second middle portion 334 may any prismatic shape. It should be understood that when first middle portion 332 and second middle portion 334 are prismatic shapes, each middle portion 332, 334 may be similar sizes to one another. First flange 331 may include a proximal facing surface 355. Sponge 330 may include a proximally extending protrusion 360 that protrudes from proximal facing surface 355 and has any of the features of protrusion 160.

In one or more embodiments, diameter 332D of first middle portion 332 and diameter 334D of second middle portion 334 may be different from one another. In some embodiments, one or more of diameters 331D, 333D, 335D of flanges 331, 333, 335, respectively, may be different than the diameters of one or more of the other flange(s). In some embodiments, diameters 331D, 333D, and 335D may each be a different value. First middle portion 332 and second middle portion 334 may have a same axial/longitudinal length or may have differing axial/longitudinal lengths. For example, second middle portion 334 may have a shorter/smaller axial/longitudinal length than first middle portion 332.

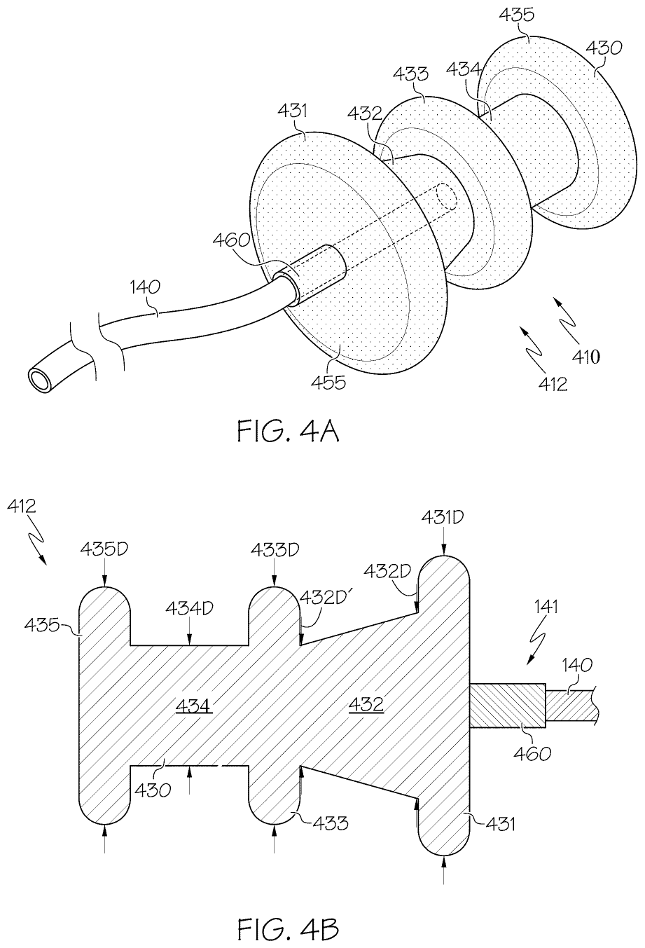

FIGS. 4A-4B depicts a distal end 412 of an exemplary medical device 410. Distal end 412 may have any of the features of distal ends 112, 312, unless otherwise specified. Where feasible, reference numbers of distal end 312 add 200 to reference numbers of distal end 112 to reference numbers of distal end 312 in order to indicate corresponding structures.

Distal end 412 may include a sponge 430 and tube 140. Sponge 430 may be positioned at distal end 141 of tube 140. Sponge 430 may include a first (proximal) flange 431, a second (middle) flange 433, and a first middle portion 432 (e.g., a saddle). First middle portion 432 may be positioned between first flange 431 and second flange 433. Sponge 430 may include a third (distal) flange 435 and a second middle portion 434 (e.g., a saddle). Second middle portion 434 may be positioned between second flange 433 and third flange 435. First flange 431 may be positioned proximally of second flange 433 and third flange 435. Third flange 435 may be positioned distally of first flange 431 and second flange 433. Second flange 433 may be between first flange 431 and third flange 435. First flange 431, second flange 433, third flange 435, first middle portion 432, and second middle portion 434 may be arranged along a longitudinal axis (e.g., a central longitudinal axis) of vacuum tube 140. In some examples, central longitudinal axes of flanges 431, 433, 435 and middle portions 432, 434 may be coaxial with a central longitudinal axis of vacuum tube 140. First flange 431 may include a proximal facing surface 455. Sponge 430 may include a proximally extending protrusion 460, having any of the features of protrusion 160.

A diameter 431D of first flange 431 may be larger than a diameter 433D of second flange 433 and may be larger than a diameter 435D of third flange 435. In some examples, diameter 433D of second flange 433 may be larger than a diameter 435D of third flange 435. Alternatively, diameter 433D of second flange 433 may be approximately equal to diameter 435D of third flange 435.

First middle portion 432 (which may be proximal of second middle portion 434) may taper from a larger diameter 432D near (e.g., adjacent to/contacting) first flange 431 to a smaller diameter 432D′ near (e.g., adjacent to/contacting) second flange 433. Smaller diameter 432D′ may be the same diameter or approximately the same diameter as a diameter 434D of second middle portion 434. Larger diameter 432D may be larger than diameter 434D of second middle portion 434. Larger diameter 432D may be smaller than diameter 433D of second flange 433. First middle portion 432 may be a frustum-shaped, however first middle portion 432 is not limited to a particular shape and may be any tapered shape. In some examples, first middle portion 432 may taper linearly. In other examples, first middle portion 432 may taper non-linearly.

In some embodiments, second middle portion 434 may taper in diameter from the proximal to distal direction or the distal to proximal direction. In such examples, second middle portion 434 may taper at a same angle as first middle portion 432, at a steeper angle, or at a shallower angle. In examples, a proximal end of second middle portion 434 may have a diameter that is equal to or approximately equal to smaller diameter 432D′ and smaller than diameter 433D of second flange 433. In some examples, middle portions 432, 434 may have approximately the same axial/longitudinal lengths. In other examples, middle portions 432, 434 may have differing axial/longitudinal lengths. For example, second middle portion 434 may have a smaller axial length than first middle portion 432.

During a procedure using distal ends 312 or 412, sponge 330, 430 may be inserted into a target cavity. For example, sponge 330, 430 may be inserted such that first middle portion 332, 432 and second middle portion 334, 434 are within the cavity. Second flange 333, 433 and third flange 335, 435 may also be received within the cavity. First flange 331, 431 may be proximal of/outside of the cavity. Over time, a size of the cavity may decrease or properties of the cavity may otherwise change. An operator may pull distal end 312, 412 proximally, such that first middle portion 332, 432 is removed from the target cavity. Distal end 312, 412 may be partially or fully retracted in a proximal direction by pulling tube 140. Alternatively, distal end 312, 412 may be partially or fully retracted in a proximal direction by pulling distal end 312, 412 with an ancillary device, such as but not limited to forceps (e.g., endoscopic rat tooth forceps). A tapered shape of first middle portion 432 of sponge 430 may assist with such repositioning of sponge 430. In some examples, second flange 333, 433 may remain within the cavity after first middle portion 332, 432 is removed. Alternatively, second flange 333, 433 may be removed from the cavity such that it is proximal of the cavity and acts as an anchor outside of the cavity. Second middle portion 334, 434 may have a smaller diameter and/or axial/longitudinal length than first middle portion 332, 432, which may accommodate a smaller size of the target cavity over time.

Although sponges 330, 430 are described as having two middle portions (e.g., saddles), it will be appreciated that sponges 330, 430 may have more than two middle portions. In some examples, further distal middle portions may have increasingly small diameters (and may have tapered shapes, as first middle portion 432 of sponge 430) to accommodate further decreases in size of the target cavity. An operator may continue to pull sponges 330, 430 distally as described above, for additional middle portions 332, 432.

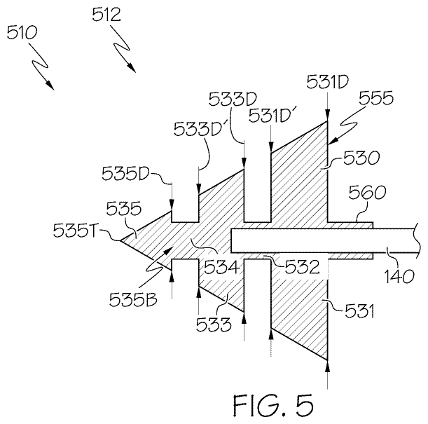

FIG. 5 depicts a cross-section of a distal end 512 of an exemplary medical device 510. Distal end 512 may include a sponge 530 and tube 140. Sponge 530 may be positioned at a distal end of tube 140. Sponge 530 may have any of the features of sponges 130, 330, 430, described above, unless otherwise specified.

Sponge 530 may include a first (proximal) flange 531, a second (middle) flange 533, and a distal tip 535. A first middle portion 532 may be positioned between first flange 531 and second flange 533, and a second middle portion 534 may be positioned between second flange 533 and distal tip 535. First flange 531 and second flange 533 may be frustum shaped, however first flange 531 and second flange 533 are not limited to being frustum shaped and may be any shape (e.g., any tapered shape), as discussed further below. First flange 531 may be positioned proximally of second flange 533 and distal tip 535. Distal tip 535 may be positioned distally of first flange 531 and second flange 533. Second flange 533 may be between first flange 531 and distal tip 535. First flange 531, second flange 533, distal tip 535, first middle portion 532, and second middle portion 534 may be arranged along a longitudinal axis (e.g., a central longitudinal axis) of vacuum tube 140. Central longitudinal axes of flanges 531, 533, distal tip 535, and middle portions 532, 534 may be approximately coaxial with a central longitudinal axis of vacuum tube 140. Although two flanges 531, 533 and a distal tip 535 are disclosed, it will be appreciated that sponge 530 may include any suitable number of flanges.

A proximal end of first flange 531 may have a maximum diameter 531D, and a proximal end of first flange 531 may have a minimum diameter 531D′. First flange 531 may taper radially inward in a distal direction, from maximum diameter 531D to minimum diameter 531D′. First flange 531 may taper linearly or non-linearly. A proximal end of second flange 533 may have a maximum diameter 533D, and a distal end of second flange 533 may have a minimum diameter 533D′. Second flange 533 may taper radially inward in a distal direction, from maximum diameter 533D to minimum diameter 533D′. Second flange 533 may taper linearly or non-linearly. First flange 531 may include a proximal facing surface 555. Sponge 530 may include a proximally extending protrusion 560 that protrudes from proximal facing surface 555. Protrusion 560 may have any of the features of protrusion 160.

Distal tip 535 may be frustum or cone shaped. However, distal tip 535 is not limited to having a frustum or cone shape and may have any shape (e.g., a tapered shape). Distal tip 535 may include a base 535B and a tip/apex 535T. Base 535B may have a circular cross-section perpendicular to a longitudinal axis of sponge 530. Base 535B may have a diameter 535D. Apex 535T may be more distal than base 535B and may be a distalmost end of sponge 530. Apex 535T may be pointed (e.g., a tip of a cone-shaped distal tip 535). In other examples, apex 535T may have a diameter that is smaller than diameter 535D of base 535B but not be pointed. Base 535B may taper linearly or non-linearly from base 535B to apex 535T.

Maximum diameter 531D of first flange 531 may be larger than maximum diameter 533D of second flange 533 and diameter 535D of base 535B. Minimum diameter 531D′ of first flange 531 may be larger than maximum diameter 533D of second flange 533, and minimum diameter 533D′ of second flange 533 may be larger than diameter 535D of base 535B. In some examples, sponge 530 may have an overall conical shape, such that a single straight line may be drawn along outer surfaces of first flange 531, second flange 533, and distal tip 535.

As compared with sponges 130, 330, 430, middle portions 532, 534 may have smaller diameters, such that outer surfaces of middle portions 532, 534 are closer to vacuum tubes than the middle portions of sponges 130, 330, 430. In some examples, as shown in FIG. 5, middle portions 532, 534 may have approximately the same diameter as protrusion 560.

When used in a procedure, sponge 530 may initially be fully inserted within a target cavity so that distal tip 535, second flange 533, and first flange 531 are fully contained within the cavity. As the target cavity (e.g. the wound) begins to heal and close up, sponge 530 may be partially removed (retracted proximally) so that only second flange 533 and distal tip 535 are within the target cavity, while first flange 531 is no longer contained in the target cavity. In some examples, this retraction of sponge 530 may occur after a predetermined time (e.g., 3-5 days) or may depend on a healing rate of the cavity. Partially removing sponge 530 may accommodate a shrinking size of the cavity so that the cavity may continue shrinking. Additionally, partially removing sponge 530 may prevent tissue from growing into sponge 530. As the cavity continues to heal and close up, sponge 530 may be further removed from cavity 151 so that only distal tip 535 remains within the cavity, while both first flange 531 and second flange 533 are outside of (proximal of) the cavity. As mentioned above, sponge 530 may include flanges in addition to first flange 531 and second flange 533, and the method above may be repeated for any number of flanges. In alternate embodiments, each flange may include a visual marker to assist in identification of the flanges, and insertion and removal of sponge 530.

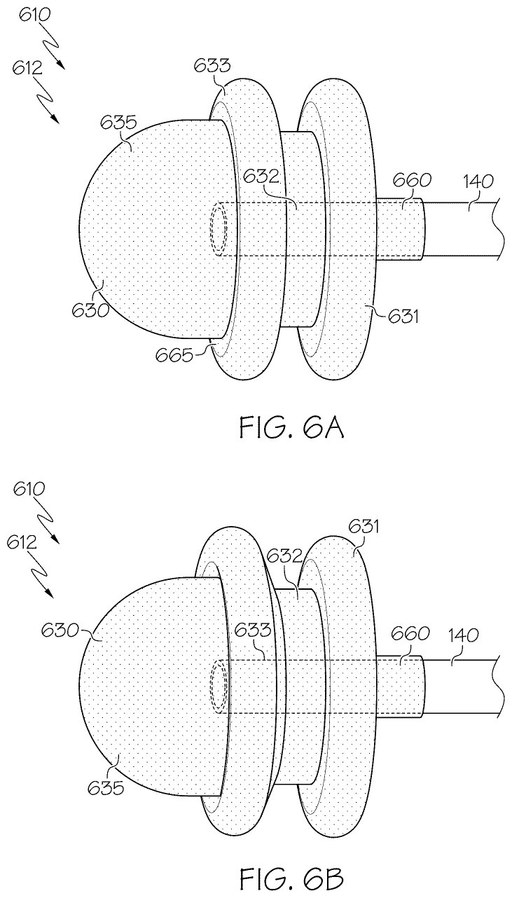

FIGS. 6A-6B depict a distal end 612 of an exemplary medical device 610. Distal end 612 may include a sponge 630 and tube 140. Sponge 630 may have any of the properties of sponges 130, 330, 430, 530, unless otherwise specified herein.

Sponge 630 may be positioned at a distal end of tube 140. Sponge 630 may include a first (proximal) flange 631, a second (distal) flange 633, and a middle portion 632 (e.g., a saddle). Middle portion 632 may be positioned between first flange 631 and second flange 633. A distal end of sponge 630 may include a protrusion 635. Protrusion 635 may protrude distally from a distal facing surface 665 of second flange 633. Protrusion 635 may form a rounded distalmost end of sponge 630. Protrusion 635 may be a portion of a sphere. For example, protrusion 635 may have a hemispherical shape, with a base of the hemisphere being proximal to a pole of the hemisphere and adjacent to second flange 633. First flange 631, second flange 633, and protrusion 635 may be arranged along a longitudinal axis (e.g., a central longitudinal axis) of vacuum tube 140. In some examples, central longitudinal axes of flanges 631, 633, middle portion 632, and protrusion 635 may be approximately coaxial with a central longitudinal axis of vacuum tube 140. First flange 631 may include a proximal facing surface. Sponge 630 may include a proximally extending protrusion 660 that protrudes from the proximal facing surface of first flange 631. Protrusion 660 may have any of the properties of protrusion 160.

Second flange 633 may be more flexible than first flange 631 (i.e., first flange 631 may be more rigid than second flange 633). For example, second flange 633 may be configured to flex in a distal and/or proximal direction. First flange 631 may be configured to be relatively rigid and not flexible in either the distal direction or the proximal direction. For example, first flange 631 may be denser than second flange 633. Additionally or alternatively, second flange 633 may have a smaller longitudinal/axial length than first flange 631 to make it more flexible. Additionally or alternatively, a shape of second flange 633 may otherwise make it more flexible than first flange 631. Second flange 633 may be more porous than first flange 631 (e.g., have larger pores or a greater number of pores). The greater porosity of second flange 633 may cause second flange 633 to be more flexible than first flange 631. Under applied negative pressure, the width/size of second flange 633 may reduce more relative to first flange 631. If both first flange 631 and second flange 633 include a hollow torus shape with a double wall (e.g., if first flange 631 and second flange 633 are hollow), with the distal flange 633 having a more porous structure compared to proximal flange 631, under the applied vacuum, the bulk of the width of the second flange 633 may reduce more when compared to first flange 631. Thus, second flange 633 may be more flexible than proximal flange 631.

For example, when used in an EVAC procedure, medical device 610 (e.g. a distal end of tube 140 and sponge 630) may be inserted into a target site (e.g. a wound within a patient's gastrointestinal tract). Second flange 633 may flex proximally as it is inserted into the cavity, to assist with insertion of sponge 630 into the cavity. Second flange 633 may be resilient, such that it may resume an unflexed shape when inserted into the cavity. Once inserted into the cavity, second flange 633 may flex in the distal direction to help to retain protrusion 635 within the target site and prevent sponge 630 from spontaneously exiting the cavity. First flange 631 may be rigid to seal or assist in sealing the cavity to prevent leakage from the cavity and to prevent debris and other contamination from entering the cavity. In aspects, second flange 633 may serve as an indicator to a user that sponge 630 is appropriately positioned within the cavity, and first flange 631 may be a retention mechanism that retains sponge 630 in a desired position.

Any of the flanges of the medical devices 110, 310, 410, 510 may be rigid or flexible, as discussed above for flanges 631, 633. In some aspects, the distalmost flange of the medical device may be flexible and the remaining flanges may be rigid. In alternate embodiments, the proximalmost flange of the medical device may be rigid and the remaining flanges may be flexible. Flexible flanges may be configured to flex only in a distal direction, only in a proximal direction, or either direction.

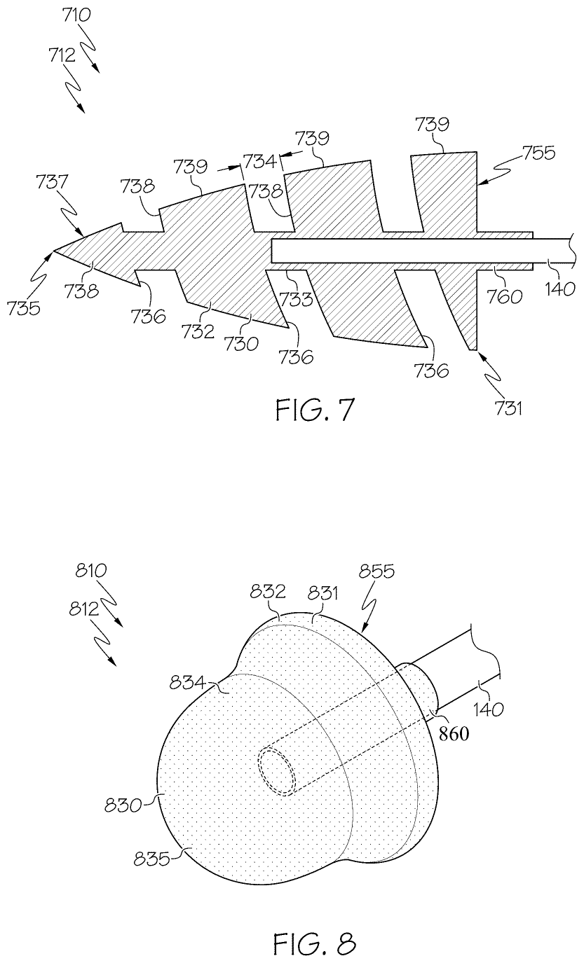

FIG. 7 depicts a distal end 712 of an exemplary medical device 710. Distal end 712 may include a sponge 730 and tube 140. Unless otherwise specified herein, sponge 730 may have any of the features of sponges 130, 330, 430, 530, 630.

Sponge 730 may be corkscrew, auger, and/or helix shaped. Sponge 730 may extend along a longitudinal axis of tube 140. Sponge 730 may include a proximal end 731 and a distal end 735. Sponge 730 may include a central core 733, which may extend from a proximal protrusion 760 (having any of the features of protrusion 160) along tube 140, to a distal tip 737 of sponge 730. Protrusion 760 may extend proximally from a proximal-facing surface 755. Proximal of distal tip 737, core 733 may have a constant diameter. As discussed below, at distal tip 737, sponge 730 may taper inward in a distal direction (e.g., to a point).

Sponge 730 may further include a sponge body 732 that may wind around core 733. Body 732 and core 733 may be a single, continuous piece of material. Body 732 may have a helical shape. Space 734 may extend between adjacent windings/turns 739 of body 732. Space(s) 734 may be any pitch and different spaces 734 of sponge 730 may have different pitches. Along spaces 734, an outer surface of core 733 may form a saddle portion between adjacent windings 739. Although FIG. 7 depicts body 732 winding around core 733 four times, it will be appreciated that any suitable number of windings/turns 739 may be used. Sponge body 732 may define a plurality of flanges that are connected to one another in a spiral shape.

An overall profile of body 732 may taper radially inward from a proximal end 731 to a distalmost end 735, such that windings 739 of body 732 become radially thinner between proximal end 731 and distal tip 737. The overall profile of body 732 may be curved, such that an outer surface of sponge 730 may be convex between proximal end 731 and distalmost end 735. In other words, a curve drawn between proximal end and distalmost end 735 that extends along a longitudinal direction and radially inward may contact outer surfaces of body 732 along each of windings 739.

Distal tip 737 of sponge 730 may taper to a point at distalmost end 735. Alternatively, distal tip 737 may have a rounded distal shape. As with more proximal portions of body 732, distal surface 738 at distal tip 737 may have a curved outer surface.

Proximal-facing surface 755 may be approximately perpendicular to a central longitudinal axis of vacuum tube 140. Proximal surfaces 736 and distal surfaces 738 of windings 739 of body 732 may be non-perpendicular to vacuum tube 140. All of proximal surfaces 736 and distal surfaces 738 of windings 739 of body 732 may have approximately the same angle with respect to the central longitudinal axis, as shown, or may have different angles. As shown in FIG. 7, proximal surfaces 736 and distal surfaces 738 of windings 739 of body 732 may be curved. For example, the proximal surface 736 of each winding may have a concave shape, and the distal surface 738 of each winding may have a convex shape.

Similarly to sponge 530, sponge 730 may be gradually removed from cavity 151 as the wound gradually heals and closes up. Initially, sponge 730 may be fully inserted into cavity 151 so proximal end 731 is within the cavity, and sponge 730 may be gradually removed from cavity 151.

FIG. 8 depicts a distal end 812 of an exemplary medical device 810. Distal end 812 may include a sponge 830 and tube 140. Unless otherwise specified, sponge 830 may have any of the properties of sponges 130, 330, 430, 530, 630, 730, described above.

Sponge 830 may be approximately mushroom- or bell-shaped. Sponge 830 may include a lip portion 832 and a dome portion 834. Lip portion 832 and dome portion 834 may be arranged along a longitudinal axis (e.g., central longitudinal axis) of tube 140. For example, central longitudinal axes of lip portion 832 and dome portion may be approximately coaxial with a central longitudinal axis of tube 140.

Sponge 830 may include a proximal end 831 and a distal end 835. Lip portion 832 may be proximal to dome portion 834. Dome portion 834 may distally protrude from lip portion 832. Dome portion 834 may be round and may be a portion of a sphere; for example, dome portion 834 may be approximately hemispherical with a base of the hemisphere being proximal to a pole of the hemisphere. The base of the hemisphere may be adjacent to lip portion 832. Dome portion 834 may taper radially outward moving proximally from proximal end 831 (and may taper radially inward moving distally toward distal end 835) Sponge 830 may include a proximal facing surface 855. Sponge 830 may include a proximally extending protrusion 860 that protrudes from proximal facing surface 855. Protrusion 860 may have any of the features of protrusion 160.

When sponge 830 is inserted into target site 150, lip portion 832 may prevent fluid from target site 150 from exiting the target site by plugging an opening of a cavity of target site 150. Lip portion 832 may serve as a visual or tactile indicator for a medical professional to seat sponge 830 within cavity 151. Lip portion 832 may be configured (sized and shaped) to prevent sponge 830 from spontaneously exiting target site 150 absent human action. Lip portion 832 is shown in FIG. 8 as being a uniform circular shape, however lip portion 832 is not limited to this shape. In alternate embodiments, lip portion 832 may include a plurality of protrusions (not shown) extending radially outward and/or proximally. The plurality of protrusions may be evenly arranged around a circumference of lip portion 832. The plurality of protrusions may protrude radially outward, proximally, or a radially outward in a proximal direction. The roundness/tapered shape of dome portion 834 may allow for easier insertion of sponge 830 within the cavity 151 of target site 150.

Although the sponges 130, 330, 430, 530, 630, 730, 830 are described as separate aspects of the disclosure, it should be understood that any of sponges 130, 330, 430, 530, 630, 730, 830 may have any of the features of the other sponges. Furthermore, any of the features of any of sponges 130, 330, 430, 530, 630, 730, 830 may be combined with any of the features of the other sponges. For example, but not to serve as a limitation, sponge 830 may be attached to a distal end of sponge 430. As another example, but not to serve as a limitation, distal tip 535 of sponge 530 may replace third flange 435 of sponge 430.

Although the sponges above are described as being fixedly attached to tubes, it will be appreciated that the sponges may be removably coupled to tubes via a connector (e.g., a quick-connect connector). Such a connector may allow for exchange of sponges during a course of treatment to accommodate a changing wound size. It should also be understood that the sponges may be pre-sutured or otherwise fixedly attached to tube 140. Further, sponges 130, 330, 430, 530, 630, 730, 830 may come in a variety of sizes to accommodate different cavity sizes and reduce the burden on surgeons to reduce the size of the sponges by other means.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A distal end of a medical device comprising:
a tube; and
a sponge positioned at a distal end of the tube;
wherein the sponge includes a first flange, a second flange, and a saddle portion positioned between the first flange and the second flange.

2. The distal end of claim 1, wherein the saddle portion is a first saddle portion, wherein the sponge includes a third flange between the first flange and the second flange, wherein the first saddle portion is between the first flange and the third flange, and wherein the sponge further includes a second saddle portion between the third flange and the second flange.

3. The distal end of claim 2, wherein a diameter of the first saddle portion is smaller than diameters of each of the first flange, the second flange, and the third flange.

4. The distal end of claim 2, wherein a diameter of the second saddle portion is smaller than diameters of each of the first flange, the second flange, and the third flange.

5. The distal end of claim 2, wherein an outer surface of the first saddle portion tapers radially inward in a distal direction.

6. The distal end of claim 2, wherein a diameter of the first flange is larger than a diameter of the second flange, and wherein the diameter of the first flange is larger than a diameter of the third flange.

7. The distal end of claim 6, wherein the diameter of the second flange is approximately equal to the diameter of the third flange.

8. The distal end of claim 1, wherein the sponge includes a rounded portion extending distally from the second flange.

9. The distal end of claim 1, wherein one of the first flange or the second flange is more flexible than the other of the first flange or the second flange.

10. The distal end of claim 1, wherein at least one of the first flange or the second flange has an outer surface that tapers radially inward in a distal direction.

11. The distal end of claim 10, further including a distal tip having an outer surface that tapers radially inward in the distal direction.

12. The distal end of claim 11, wherein a distalmost end of the distal tip is pointed.

13. The distal end of claim 12, wherein a same line extends along outer surfaces of the first flange, the second flange, and the distal tip.

14. The distal end of claim 1, wherein the first flange and the second flange are connected to form a helix.

15. The distal end of claim 14, wherein a single curve extends along a first surface surfaces and a second surface of the helix.

16. A distal end of a medical device comprising:
a tube; and
a sponge positioned at a distal end of the tube;
wherein the sponge includes a flange and a portion extending distally of the flange, wherein an outer surface of the portion extending distally of the flange tapers radially inward in a distal direction.

17. The distal end of claim 16, wherein the flange is a first flange, the distal end further comprising a second flange that is distal of the first flange, wherein the outer surface of the portion extending distally of the first flange tapers between the first flange and the second flange.

18. The distal end of claim 17, wherein the outer surface of the portion extending distally of the first flange has a rounded shape, forming a rounded distalmost end of the sponge.

19. The distal end of claim 17, wherein the outer surface of the portion extending distally of the first flange is an outer surface of the second flange, wherein a saddle portion extends between the first flange and the second flange.

20. A medical method comprising:
moving a sponge coupled to a distal end of a tube proximally, such that the sponge transitions from a first configuration, in which a first flange and a second flange of the sponge are within a cavity of a body lumen and a third flange of the sponge is outside of the cavity, to a second configuration, in which the first flange is within the cavity of the body lumen, and the second flange and the third flange are outside of the cavity; and
applying a negative pressure to the sponge using the tube.
